(19) United States
(12) Patent Application Publication
(10) Pub. No.: US 2014/0114207 A1
(43) Pub. Date: Apr. 24, 2014

(54) Cognitive Management Method and System

(71) Applicant: Timothy Patterson, Pleasanton, CA (US)

(72) Inventor: Timothy Patterson, Pleasanton, CA (US)

(21) Appl. No.: 13/655,417

(22) Filed: Oct. 18, 2012

(51) Int. Cl. A61B 5/0482; A61B 5/0484

(52) U.S. Cl. 600/545; 600/544

(57) ABSTRACT

A cognitive management method and system that monitors and manipulates a response signal from a neurological organ to optimize cognitive performance and behavioral management of the neurological organ. The method allows a user to transmit a stimulus signal such as optical or auditory to the organ and receive a return signal in response. A sensor, such as EEG electrodes detects the response signal. The response signal generated by the neurological organ is then detected by the sensor and transmitted to data storage. The signal can be reconfigured and retransmitted to a display device. The signal includes Hertz and real time frequencies. The signal includes a signal parameter with a high range and a low range. An alert actuates when the signal exceeds the parameter. The signal is monitored by the user and invited followers to analyze and treat cognitive performance and behavior.

### CROSS-REFERENCE TO RELATED APPLICATIONS

The present Utility patent application claims priority benefit of the U.S. provisional application for patent Ser. No. 61/548,723 entitled “Method for monitoring and managing neuro-electrophysiological activity associated with cognitive performance and behavioral management”, filed on Oct. 19, 2011 under 35 U.S.C. 119(e). The contents of this related provisional application are incorporated herein by reference for all purposes to the extent that such subject matter is not inconsistent herewith or limiting hereof.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to cognitive states. More particularly, one or more embodiments of the invention relate to a method of monitoring and manipulating cognitive states.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that cognition is a group of mental processes that includes attention, memory, producing and understanding language, solving problems, and making decisions. Cognition is studied in various disciplines such as psychology, philosophy, linguistics, science and computer science.

Typically, cognitive science is the interdisciplinary scientific study of the mind and its processes. Cognitive science examines what cognition is, what it does and how it works. It includes research on intelligence and behavior, especially focusing on how information is represented, processed, and transformed in faculties such as perception, language, memory, reasoning, and emotion within the brain and nervous system.

Typically, electroencephalography (EEG) is the recording of electrical activity along the scalp. EEG provides stimulus and measures voltage fluctuations resulting from ionic current flows within the neurons of the brain.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a flowchart diagram of the steps for an exemplary cognitive management method, in accordance with an embodiment of the present invention;

FIG. 2 illustrates a block diagram for an exemplary cognitive management system, in accordance with an embodiment of the present invention;

FIGS. 3A, 3B, 3C, and 3D illustrate top views of an exemplary display portion for an exemplary user, where FIG. 3A illustrates an exemplary general information display, FIG. 3B illustrates an exemplary user information display, FIG. 3C illustrates an exemplary follower list display, and FIG. 3D illustrates an exemplary message display, in accordance with an embodiment of the present invention;

FIG. 4 illustrates a top view of an exemplary display portion for an exemplary follower, in accordance with an embodiment of the present invention;

FIG. 5 illustrates an exemplary Delta neurological organ frequency, in accordance with an embodiment of the present invention;

FIG. 6 illustrates an exemplary Theta neurological organ frequency, in accordance with an embodiment of the present invention;

FIG. 7 illustrates an exemplary Alpha neurological organ frequency, in accordance with an embodiment of the present invention;

FIG. 8 illustrates an exemplary Beta neurological organ frequency, in accordance with an embodiment of the present invention; and

FIG. 9 illustrates a block diagram depicting an exemplary client/server system which may be used by an exemplary web-enabled/networked embodiment of the present invention, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present application or of any further application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

A non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; however, the non-transitory computer readable medium does not include a pure transitory signal per se.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

There are various types of cognitive management methods and systems that may be provided by preferred embodiments of the present invention. In one embodiment of the present invention, the cognitive management method may allow a user to monitor and manipulate a neurological organ for the effect of producing optimal cognitive performance and behavior management in the neurological organ. The cognitive management method may allow a user to transmit a stimulus to the neurological organ for manipulating the neurological organ and recording a response signal from the neurological organ. The response signal may include a neurological organ frequency that represents various cognitive states. The cognition may include, without limitation, knowledge, memory, judgment, reasoning, and mental processes of perception. In one embodiment of the present invention, the neurological organ may include, without limitation, the brain, the spine, and the nervous system. However, in other embodiments, any organ that produces a physical or electrophysiological signal may be monitored and manipulated by the cognitive management method. In this manner, the user may better comprehend the cognitive state, and make corrective actions, including, without limitation, providing stimulus to manipulate the neurological organ, receiving medicine, and changing lifestyle habits.

In one embodiment of the present invention, the stimulus signal applied to the neurological organ may include, without limitation, audio signals, isochronic tones, binaural beats, filtered noise and background sounds, visual pulses, electrophysiological signals, and electrical pulses. However, in other embodiments, the stimulus signal may include a rhythmic stimulus that facilitates recognition by the brain. The rhythmic stimulus may include, without limitation, a drum beat, wherein the brain may pattern a response to match the rhythm, including, without limitation, a Frequency Following Response (FFR). In this manner, the brain may be manipulated to generate a desired response. In some embodiments, the response signal from the neurological organ may include, without limitation, an electrical signal, a cortical evoked response, brainwave frequencies in Hertz, and voltage fluctuations from ionic current flows. In this manner, the neurological organ may generate a neurological organ frequency. In some embodiments, the neurological organ frequency may include four frequency bands, or brainwave frequencies, including, without limitation Delta 0.2-3 Hertz for sleep, Theta 3-8 Hertz for creativity, Alpha 8-12 Hertz for relaxation, and Beta 12-38 Hertz for concentration. In this manner, the neurological organ may receive the stimulus to generate the desired cognitive state. Those skilled in the art, in light of the present teachings will recognize that the cognitive state may also be monitored for various purposes, including, without limitation, therapy, scientific study, refocusing, and relaxation. In some embodiments, the user or a follower may preset a target frequency for the response signal. If the target exceeds a predetermined amount, an alert may be activated.

In one embodiment of the present invention, the cognitive management method may monitor the response signal generated by the neurological organ. The response signal may include the neurological organ frequency in Hertz. Those skilled in the art, in light of the present teachings will recognize that when a brain is given a stimulus, through the ears, eyes or other senses, the brain may generate a response that includes an electrical impulse in the form of a cortical evoked response. The cortical evoked response may travel throughout the brain to form tangible sensory functions, including, without limitation, sight, auditory reception, tactility, and smell. In one embodiment, the cortical evoked response may be measured using electroencephalography (EEG) technology with electrodes attached to a scalp.

In some embodiments, a sensor portion may receive the response signal from the neurological organ. However, in other embodiments, the sensor portion may stimulate the neurological organ for manipulating to a desired neurological organ frequency. The sensor portion may join in proximity to the neurological organ to record a more accurate response. The sensor portion may include, without limitation, electrodes, EEG recording nets, and circuits. The sensor portion may be configured to transmit readings from the signal to a data storage portion. The data storage portion may be remotely located in relation to the sensor portion. The data storage portion may include, without limitation, a database, a processor, a network, and a cloud. In some embodiments, the data storage portion may be operable to transform the signal from one form to another.

In one embodiment of the present invention, the data storage portion may transmit the signal to a display portion. The display portion may include, without limitation, a mobile electronic device, a smart phone, a computer monitor, and a tablet. The display portion may display the signal that correlates to the neurological organ. The display portion may include a signal parameter that displays a high range and a low range acceptable for the signal of the neurological organ. In some embodiments, the display portion may alert the user if the signal reaches a predetermined level. In this manner, the user may be informed if the neurological organ is not functioning properly, is functioning properly, is healing, or requires additional attention. The display portion may further include the identification of the user. The display portion may further include a date and time stamp indicating the last period that the user was physically examined, or the last time the signal was monitored. A follower may also receive the signal from a different display portion. The follower may include, without limitation, a health care professional and a family member. In one embodiment, the follower may also be alerted if the signal reaches a predetermined level to know if the neurological organ is not functioning properly. In this manner, the follower may assist the user. The follower may monitor a plurality of users from the display portion. In yet another embodiment, the follower and the user may interchange messages pertinent to the neurological monitoring method through the respective display portions. In yet another embodiment, the follower may set a predetermined parameter for the signal. If the parameter is exceeded, the display portion may alert the follower to take corrective actions.

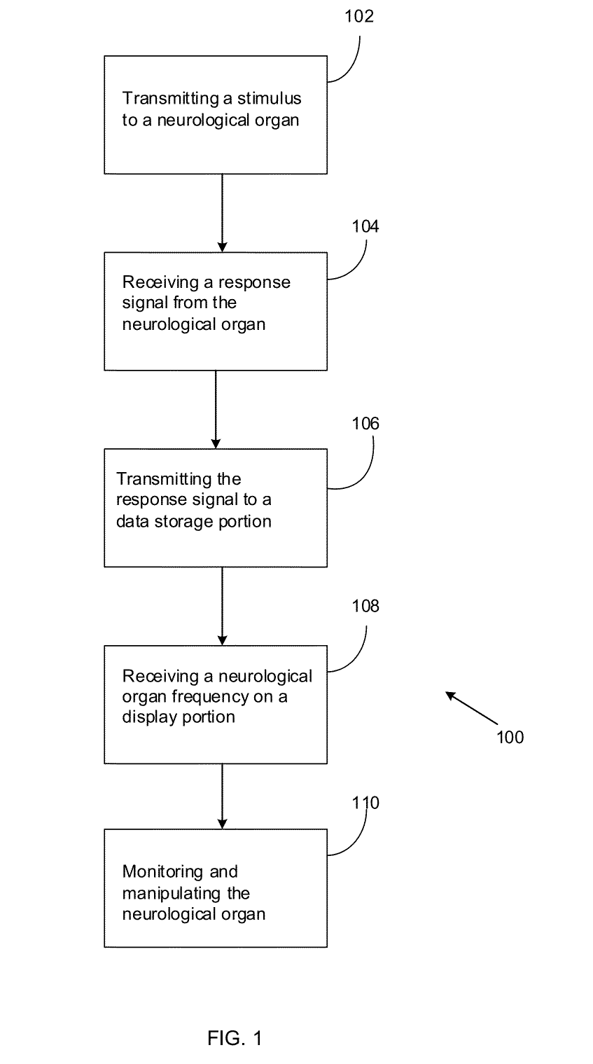

FIG. 1 illustrates a flowchart diagram of the steps for an exemplary cognitive management method 100, in accordance with an embodiment of the present invention. In the present embodiment, the cognitive management method may include a Step 102 of transmitting a stimulus signal to the neurological organ. The stimulus signal may include the neurological organ frequency in Hertz. In some embodiments, at least one sensor portion may be utilized to transmit the stimulus. In some embodiments, the at least one sensor portion may join in proximity to an affected area to provide a more accurate reading of the neurological organ. The sensor portion may include, without limitation, electrodes, EEG recording nets, conductive gel, and circuits. In one embodiment, transmitting the stimulus to the neurological organ may include, without limitation, recording electrical activity along the scalp with electrodes and a conductive gel positioned on the scalp, and transmitting an electrical rhythmic pulse. Various methods for transmitting the stimulus may be utilized including, without limitation, electroencephalography (EEG), functional magnetic resonance imaging (fMRI), positron emission tomography, magnetoencephalography, nuclear magnetic resonance spectroscopy, electrocorticography, and single-photon emission computed tomography. The sensor portion may be configured to transmit readings from the response signal of the neurological organ to the data storage portion.

Those skilled in the art, in light of the present teachings will recognize that when a brain is stimulated with the stimulus, through the ears, eyes or other senses, the brain may generate a response that includes an electrical impulse in the form of a cortical evoked response. The cortical evoked response may travel throughout the brain to form tangible sensory functions, including, without limitation, sight, auditory reception, tactility, and smell. In one embodiment, the cortical evoked response may be measured using electroencephalography (EEG) technology with electrodes attached to a scalp.

In one embodiment of the present invention, the neurological monitoring method allows a user to monitor and manipulate the neurological organ that generates a physical or electrophysiological signal in response to the stimulus. In this manner, the user may better comprehend the cognitive state, and make corrective actions, including, without limitation, providing stimulus to manipulate the neurological organ, receiving medicine, and changing lifestyle habits.

In one alternative embodiment, an injury to the neurological organ may also be monitored by studying the cognitive state of the user. In other embodiments, the neurological monitoring method may allow a user to recover from the injury, map the neurological organ, and study the neurological organ. The injury may include, without limitation, a mild traumatic brain injury, a concussion, a hemorrhage, and a headache.

In one embodiment of the present invention, Step 104 may include receiving a response signal from the neurological organ. The cognitive management method may monitor the response signal generated by the neurological organ. The response signal may include the neurological organ frequency in Hertz. Those skilled in the art, in light of the present teachings will recognize that when a brain is given a stimulus, through the ears, eyes or other senses, the brain may generate a response that includes an electrical impulse in the form of a cortical evoked response. The cortical evoked response may travel throughout the brain to form tangible sensory functions, including, without limitation, sight, auditory reception, tactility, and smell. In one embodiment, the cortical evoked response may be measured using electroencephalography (EEG) technology with electrodes attached to a scalp.

In one embodiment of the present invention, Step 106 may include transmitting the response signal to a data storage portion. The sensor portion may be configured to transmit readings from the signal to a data storage portion. The data storage portion may be remotely located in relation to the sensor portion. The data storage portion may include, without limitation, a database, a processor, a network, and a cloud. In some embodiments, the data storage portion may be operable to transform the signal from one form to another.

In one embodiment of the present invention, Step 108 may include receiving the response signal on a display portion. The display portion may include, without limitation, a mobile electronic device, a smart phone, a computer monitor, and a tablet. The display portion may display the signal that correlates to the neurological organ. The display portion may include a signal parameter that displays a high range and a low range acceptable for the signal of the neurological organ. In some embodiments, the display portion may alert the user if the signal reaches a predetermined level. In this manner, the user may be informed if the neurological organ is not functioning properly, is functioning properly, is healing, or requires additional attention. The display portion may further include the identification of the user. The display portion may further include a date and time stamp indicating the last period that the user was physically examined, or the last time the signal was monitored. A follower may also receive the signal from a different display portion. The follower may include, without limitation, a health care professional and a family member.

In one embodiment of the present invention, Step 110 may include monitoring and manipulating the resultant response signal from the neurological organ. The display portion may allow the user and the follower to simultaneously monitor the status of the neurological organ to better comprehend the cognitive state of the user. Further, the user and follower may manipulate the neurological organ by applying the stimulus to the neurological organ to generate a desired cognitive state. For example, without limitation, a student performing normally for their age and academic level may have a general range of time for an attention span. As the student approach the end of the attention span range, the student may require a short break to refocus attention. Generally taking a few minutes to refresh and refocus, the student's brain may be centering on a certain neuro-electrophysiological band. The four generally recognized bands (brainwave frequencies) are Delta 0.2-3 Hz (sleep), Theta 3-8 Hz (creativity), Alpha 8-12 Hz (relaxation), Beta 12-38 Hz (concentration). The ability to refocus varies from person to person with easily highly-focused on one end of the range and absolute inability on the other end of the range. Therefore, monitoring the effectiveness of medical protocols and/or stimulus manipulation as it relates to cognitive performance may allow the user to better understand the cognitive state and make necessary corrective action, including, without limitation, providing a stimulus to the brain, medication, and changing lifestyle habits.

Those skilled in the art will recognize that the display portion may be configured to display only pertinent information regarding the response signal and the neurological organ frequency. In some embodiments, the follower may receive and view the response signal from a different display portion simultaneously with the user. The follower may include, without limitation, a health care professional, a coach, a psychiatrist, and a family member. In one embodiment, the follower may also be alerted if the signal reaches a predetermined level to know if the cognitive state and neurological organ are not functioning properly. In some embodiments, the predetermined level may include a target level appropriate for the situational conditions being monitored. In this manner, the follower may assist or study the user's neurological organ and behavior. The follower may monitor a plurality of users from the display portion. In yet another embodiment, the follower and the user may interchange messages pertinent to the neurological monitoring method through the respective display portions. In yet another embodiment, the follower may set a predetermined parameter for the response signal. If the parameter is exceeded, the display portion may alert the follower to take corrective actions. In one embodiment, the user may set upper and lower frequency alarm levels for notifying the user if the response signal, including, without limitation, EEG brainwaves, move outside the signal parameter, or user set alarm thresholds. In some embodiments, the alarm may include, without limitation, text or instant messaging notification services to other parties that notify of alarm triggers or status change. In one embodiment of the present invention, the user may monitor the response signal from a non-cloud based application. In yet another application, the display portion may include, without limitation, a smartphone with Bluetooth connectivity.

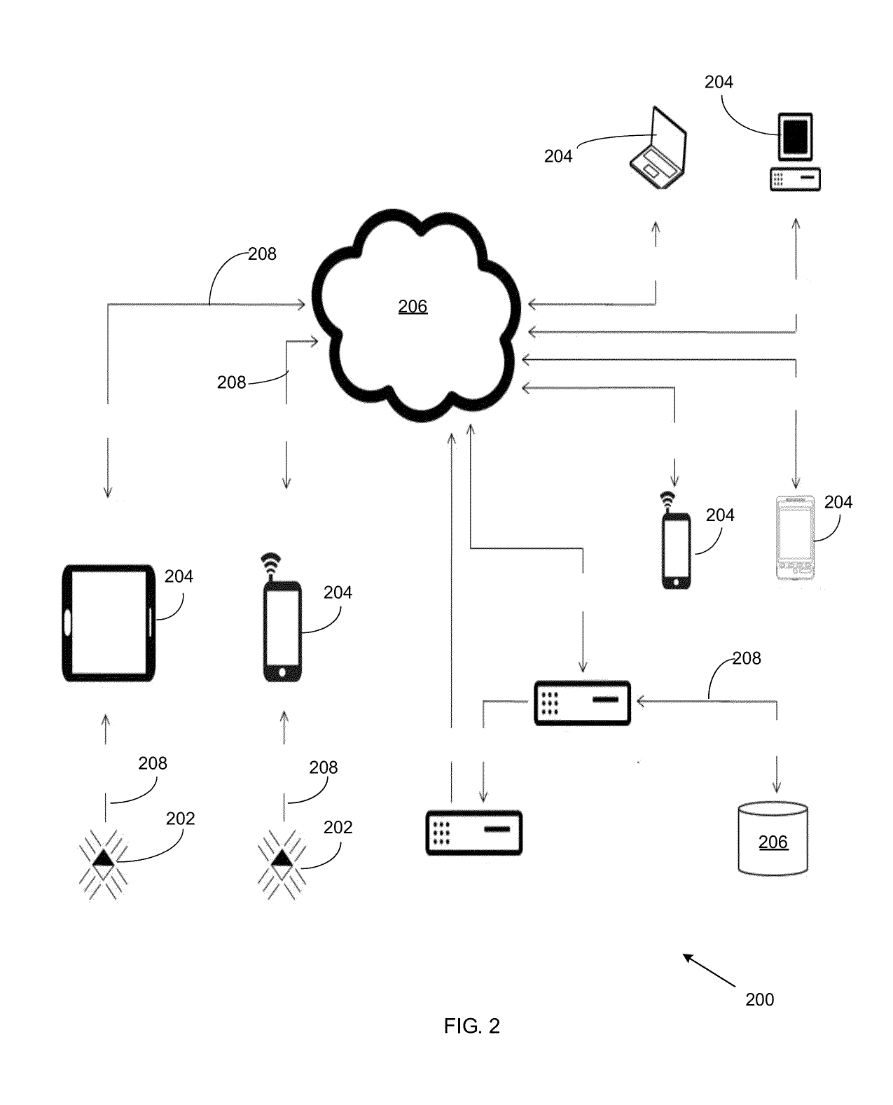

FIG. 2 illustrates a block diagram for an exemplary cognitive management system 200, in accordance with an embodiment of the present invention. In the present embodiment, the cognitive management system may include numerous systems and components working in conjunction to provide a system for monitoring and manipulating the neurological organ frequency. The sensor portion 202 may join in proximity to the neurological organ to sense for response signals 208 that the neurological organ emits. In some embodiments, the sensor portion may also stimulate the neurological organ by generating the stimulus. Those skilled in the art, in light of the present teachings will recognize that voltage fluctuations resulting from ionic current flows within the neurons of the brain emit to form response signals. The response signal may include, without limitation, electrophysiological signals, electrical pulses, voltage fluctuations from ionic current flows, and frequencies of Hertz per second. A display portion 204 may provide a visual confirmation and analysis of the response signal. The sensor portion may be configured to transmit readings from the response signal to a data storage portion 206. The data storage portion may be remotely located in relation to the sensor portion. The data storage portion may include, without limitation, a database, a processor, a network, and a cloud. In some embodiments, the data storage portion may be operable to transform the response signal from one form to another.

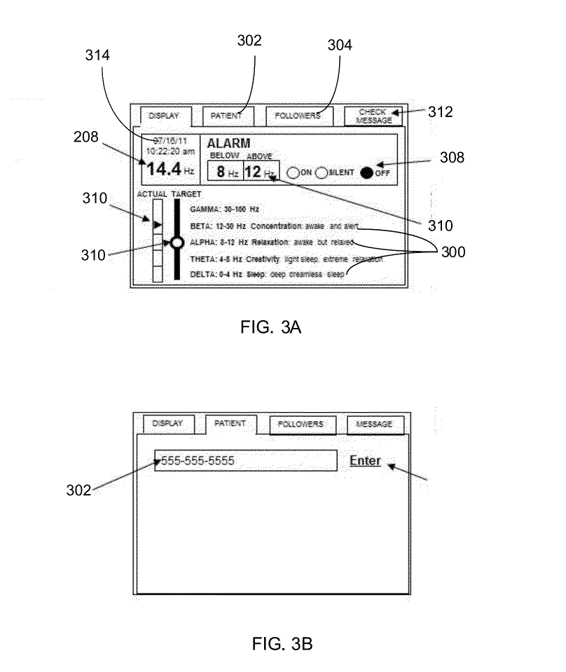

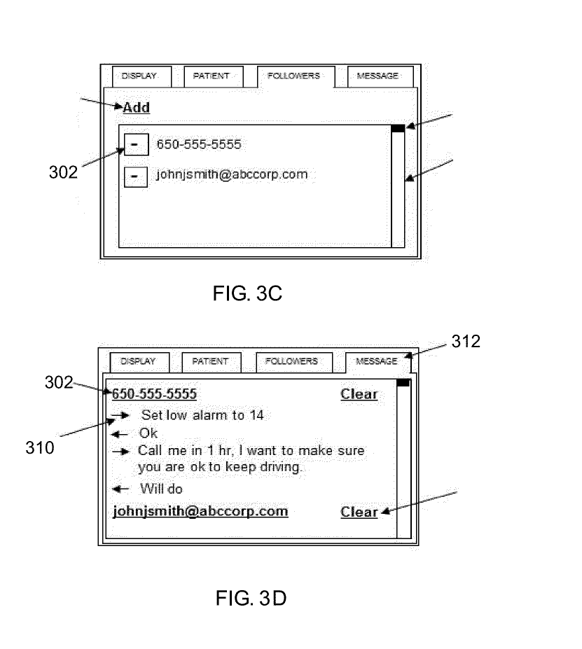

FIGS. 3A, 3B, 3C, and 3D illustrate top views of an exemplary display portion for an exemplary user, where FIG. 3A illustrates an exemplary general information display, FIG. 3B illustrates an exemplary user information display, FIG. 3C illustrates an exemplary follower display, and FIG. 3D illustrates an exemplary message display, in accordance with an embodiment of the present invention. In the present embodiment, the user may monitor the response signals emanating from the neurological organ in real time on the display portion. In this manner, at least one cognitive parameter 300 may be monitored and manipulated to help optimize cognitive performance and behavioral management. The display portion may include, without limitation, a mobile electronic device, a smart phone, a computer monitor, and a tablet. The display portion may display the response signal that correlates to the neurological organ. The display portion may include a signal parameter 310 that displays a high range and a low range acceptable for the response signal of the neurological organ. In some embodiments, the display portion may include an alert portion 308 that alerts a user if the response signal reaches a predetermined level. In this manner, the user may be informed of the neurological organ frequency, whether the neurological organ is not functioning properly, is functioning properly, is healing, or requires additional attention. The behavior of the user may also be better understood by noting when the signal parameter exceeds a predetermined mark. The display portion may further include the identification of the user 302. The display portion may further include a date and time portion 314 indicating the last period that the user was physically examined, or the last time the signal was monitored. The display portion may further include a message portion 312 so that a user and a follower 304 may communicate with each other while the signal is transmitting.

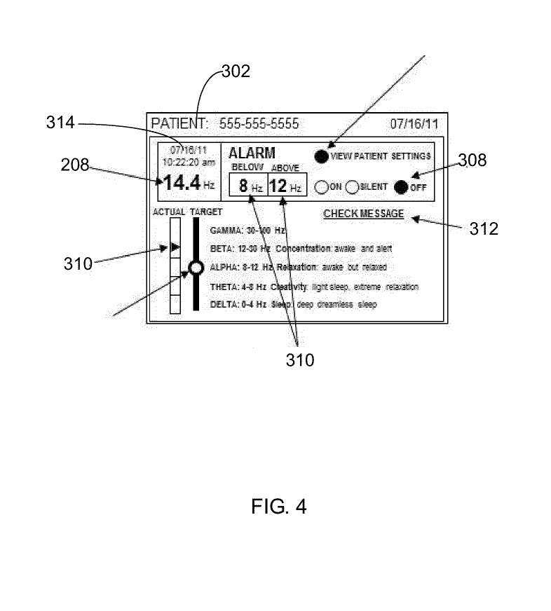

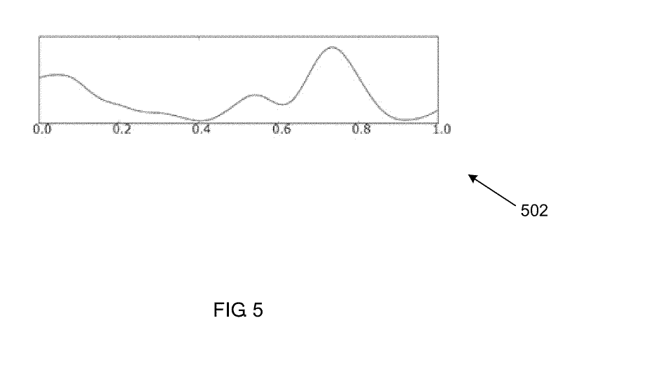

FIG. 4 illustrates a top view of an exemplary display portion for an exemplary follower, in accordance with an embodiment of the present invention. In the present embodiment, the follower may receive the response signal from a different display portion simultaneously with the user. In this manner, the follower may better monitor the neurological organ's status and the user's behavioral patterns. The follower may set the alert portion to alert if the parameters are exceeded. The follower may further set the alert portion so that only the follower is aware of the neurological organ frequency. In this manner, the user may not be stressed. In one embodiment, the follower may also be alerted if the response signal reaches a predetermined level to know if the neurological organ is not functioning properly. In this manner, the follower may assist the user. The follower may monitor a plurality of users from the display portion. In yet another embodiment, the follower and the user may interchange messages pertinent to the neurological monitoring method through the respective display portions. In yet another embodiment, the follower may set a predetermined parameter for the response signal. If the parameter is exceeded, the display portion may alert the follower to monitor the neurological organ frequency or take corrective actions. In yet another embodiment, the user may set upper and lower frequency alarm levels which may be efficacious in notifying the user if the EEG brainwaves move outside the signal parameter.

FIG. 5 illustrates an exemplary Delta neurological organ frequency 502, in accordance with an embodiment of the present invention. In the present embodiment, the Delta neurological organ frequency may include the frequency range between 0.2 Hertz and 4 Hertz. The Delta frequency may include a cognitive state of dreamless sleep. Those skilled in the art will recognize that the Delta neurological organ frequency may include the slowest band of brainwaves. When the dominant brainwave is Delta, the user may be healing resetting an internal clock. The user may not dream in the Delta neurological organ frequency and may be completely unconscious. Those skilled in the art, in light of the present teachings will recognize that any known frequencies or frequencies to be developed in the future may be monitored.

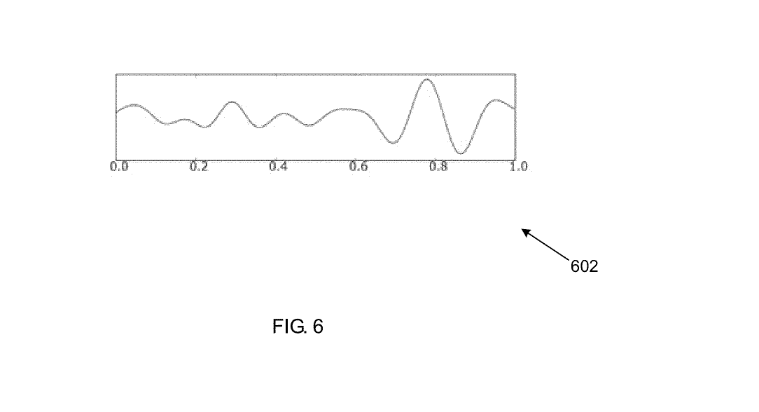

FIG. 6 illustrates an exemplary Theta neurological organ frequency 602, in accordance with an embodiment of the present invention. In the present embodiment, the Theta neurological organ frequency may include the frequency range between 4 Hertz and 7 Hertz. The Theta neurological organ frequency may include a cognitive state of creativity. Those skilled in the art will recognize that the Theta neurological organ frequency may include relaxed, meditative, and creative cognitive states. Theta neurological organ frequency may also be used for hypnosis and self-programming.

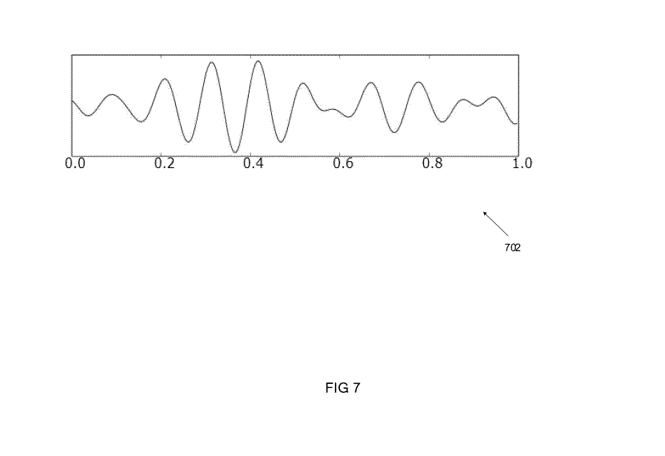

FIG. 7 illustrates an exemplary Alpha neurological organ frequency 702, in accordance with an embodiment of the present invention. In the present embodiment, the Alpha neurological organ frequency may include the frequency range between 8 Hertz and 12 Hertz. The Alpha neurological organ frequency may include a cognitive state of being awake, but relaxed, and not processing information. Those skilled in the art will recognize that the Alpha neurological organ frequency may include the state of rising in the morning and just before sleep. When the user closes the eyes the brain may automatically start producing more Alpha neurological organ frequency. In some embodiments, the Alpha neurological organ frequency may include the goal of experienced meditators, being a receptive state for effective self-hypnosis and mental re-programming.

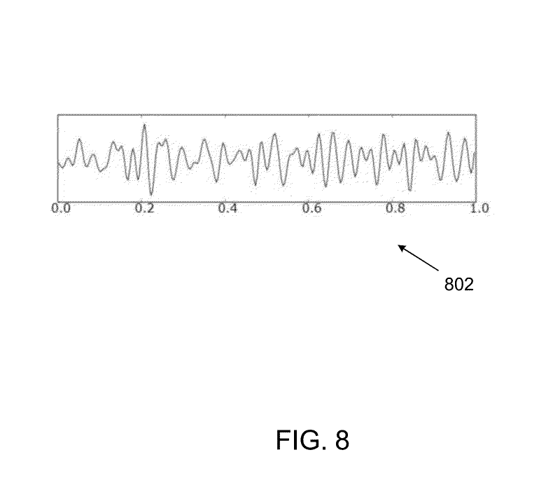

FIG. 8 illustrates an exemplary Beta neurological organ frequency 802, in accordance with an embodiment of the present invention. In the present embodiment, the Beta neurological organ frequency may include the frequency range between 12 Hertz and 30 Hertz. The Beta neurological organ frequency may include a cognitive state of being wide awake. Those skilled in the art will recognize that many people may lack sufficient Beta activity, which may result in mental or emotional disorders such as depression and insomnia. Stimulating Beta activity with the cognitive management method and system may enhance emotional stability, energy levels, attentiveness and concentration.

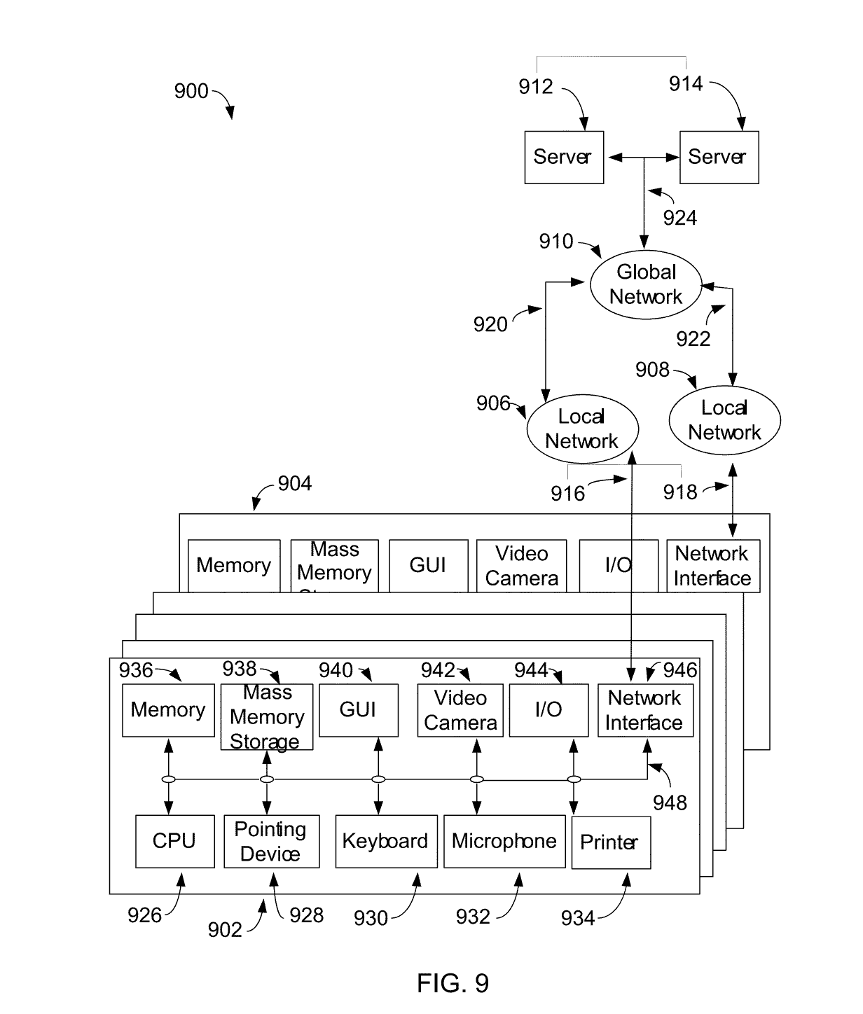

FIG. 9 illustrates a block diagram depicting an exemplary client/server system which may be used by an exemplary web-enabled/networked embodiment of the present invention. In the present embodiment, a communication system 900 includes a multiplicity of clients with a sampling of clients denoted as a client 902 and a client 904, a multiplicity of local networks with a sampling of networks denoted as a local network 906 and a local network 908, a global network 910 and a multiplicity of servers with a sampling of servers denoted as a server 912 and a server 914.

Client 902 may communicate bi-directionally with local network 906 via a communication channel 916. Client 904 may communicate bi-directionally with local network 908 via a communication channel 918. Local network 906 may communicate bi-directionally with global network 910 via a communication channel 920. Local network 908 may communicate bi-directionally with global network 910 via a communication channel 922. Global network 910 may communicate bi-directionally with server 912 and server 914 via a communication channel 924. Server 912 and server 914 may communicate bi-directionally with each other via communication channel 924. Furthermore, clients 902, 904, local networks 906, 908, global network 910 and servers 912, 914 may each communicate bi-directionally with each other.

In one embodiment, global network 910 may operate as the Internet. It will be understood by those skilled in the art that communication system 900 may take many different forms. Non-limiting examples of forms for communication system 900 include local area networks (LANs), wide area networks (WANs), wired telephone networks, wireless networks, or any other network supporting data communication between respective entities.

Clients 902 and 904 may take many different forms. Non-limiting examples of clients 902 and 904 include personal computers, personal digital assistants (PDAs), cellular phones and smartphones.

Client 902 includes a CPU 926, a pointing device 928, a keyboard 930, a microphone 932, a printer 934, a memory 936, a mass memory storage 938, a GUI 940, a video camera 942, an input/output interface 944 and a network interface 946.

CPU 926, pointing device 928, keyboard 930, microphone 932, printer 934, memory 936, mass memory storage 938, GUI 940, video camera 942, input/output interface 944 and network interface 946 may communicate in a unidirectional manner or a bi-directional manner with each other via a communication channel 948. Communication channel 948 may be configured as a single communication channel or a multiplicity of communication channels.

CPU 926 may be comprised of a single processor or multiple processors. CPU 926 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 936 is used typically to transfer data and instructions to CPU 926 in a bi-directional manner. Memory 936, as discussed previously, may include any suitable computer-readable media, intended for data storage, such as those described above excluding any wired or wireless transmissions unless specifically noted. Mass memory storage 938 may also be coupled bi-directionally to CPU 926 and provides additional data storage capacity and may include any of the computer-readable media described above. Mass memory storage 938 may be used to store programs, data and the like and is typically a secondary storage medium such as a hard disk. It will be appreciated that the information retained within mass memory storage 938, may, in appropriate cases, be incorporated in standard fashion as part of memory 936 as virtual memory.

CPU 926 may be coupled to GUI 940. GUI 940 enables a user to view the operation of computer operating system and software. CPU 926 may be coupled to pointing device 928. Non-limiting examples of pointing device 928 include computer mouse, trackball and touchpad. Pointing device 928 enables a user with the capability to maneuver a computer cursor about the viewing area of GUI 940 and select areas or features in the viewing area of GUI 940. CPU 926 may be coupled to keyboard 930. Keyboard 930 enables a user with the capability to input alphanumeric textual information to CPU 926. CPU 926 may be coupled to microphone 932. Microphone 932 enables audio produced by a user to be recorded, processed and communicated by CPU 926. CPU 926 may be connected to printer 934. Printer 934 enables a user with the capability to print information to a sheet of paper. CPU 926 may be connected to video camera 942. Video camera 942 enables video produced or captured by user to be recorded, processed and communicated by CPU 926.

CPU 926 may also be coupled to input/output interface 944 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 926 optionally may be coupled to network interface 946 which enables communication with an external device such as a database or a computer or telecommunications or internet network using an external connection shown generally as communication channel 916, which may be implemented as a hardwired or wireless communications link using suitable conventional technologies. With such a connection, CPU 926 might receive information from the network, or might output information to a network in the course of performing the method steps described in the teachings of the present invention.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC §112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC §112 (6). In some embodiments, the methods and/or system components which may be located and/or performed remotely include, without limitation all of the foregoing methods and/or system.

It is noted that according to USA law, all claims must be set forth as a coherent, cooperating set of limitations that work in functional combination to achieve a useful result as a whole. Accordingly, for any claim having functional limitations interpreted under 35 USC §112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC §112 (6) is/(are) dependent on one or more remote servers located outside the USA, it is intended that each such recited function under 35 USC §112 (6) is to be interpreted as the function of the local system receiving the remotely generated information required by a locally implemented claim limitation, wherein the structures and or steps which enable, and breath life into the expression of such functions claimed under 35 USC §112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC §112 (6)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing a method for monitoring neurological activities according to the present invention will be apparent to those skilled in the art. Various aspects of the invention have been described above by way of illustration, and the specific embodiments disclosed are not intended to limit the invention to the particular forms disclosed. The particular implementation of the method for monitoring neurological activities may vary depending upon the particular context or application. By way of example, and not limitation, the a method for monitoring neurological activities described in the foregoing were principally directed to monitoring brain activity; however, similar techniques may instead be applied to other organs of the body, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. One or more computer storage media storing computer-usable instructions, that when used by one or more computing devices, cause the one or more computing devices to perform a method comprising the steps of:
(a) transmitting a stimulus signal to a neurological organ;
(b) receiving a response signal from said neurological organ;
(c) transmitting said response signal to a data storage portion;
(d) receiving said response signal on a display portion; and
(e) monitoring and manipulating said response signal to optimize at least one cognitive parameter of said neurological organ.

2. The method of claim 1, wherein said at least one cognitive parameter comprises cognitive performance and behavior.

3. The method of claim 1, in which said neurological organ comprises a brain.

4. The method of claim 1, in which said stimulus signal comprises a rhythmic stimulus, said rhythmic stimulus comprising Hertz.

5. The method of claim 4, in which step (a) further comprises transmitting a sound or light stimulus signal for auditory or optical input and cognitive frequency influence

6. The method of claim 5, in which step (b) further comprises utilizing at least one electroencephalography electrode sensor to detect said cognitive frequency response signal.

7. The method of claim 1, in which said response signal comprises an electrical impulse, said electrical impulse comprising a cortical evoked response.

8. The method of claim 1, in which said response signal further comprises a graphical wave form display.

9. The method of claim 1, in which said response signal further comprises an alpha numeric value.

10. The method of claim 9, in which step (c) further comprises transmitting said response signal with a conventional communication protocol.

11. The method of claim 10, in which step (d) further comprises displaying said response signal on a display portion, said display portion comprising a portable electronic device.

12. The method of claim 11, in which said display portion comprises a signal parameter, said signal parameter comprising a high range and a low range.

13. The method of claim 12, in which said signal parameter comprises an alert portion, said alert portion being operable to monitor at least one cognitive parameter, said alert portion comprising audible, visible and/or physical indicators.

14. The method of claim 13, in which step (e) further comprises monitoring a real time frequency, said real time frequency comprising about 1 to 10 seconds, said real time frequency further comprising a periodic average, said periodic average comprising about 10 to 60 seconds.

15. The method of claim 14, in which step (e) further comprises a user, said user comprising a patient.

16. The method of claim 15, in which step (e) further comprises a follower, said follower comprising a medical professional.

17. The method of claim 16, in which only said user monitors said display portion with a non-cloud based application.

18. The method of claim 17, in which said user monitors said response signal from a smart phone with Bluetooth connectivity.

19. A system for monitoring comprising:
means for transmitting a stimulus signal to a neurological organ;
means for receiving a response signal from said neurological organ;
means for transmitting said response signal to a data storage portion;
means for receiving said response signal on a display portion; and
means for monitoring and manipulating said response signal to optimize at least one cognitive parameter of said neurological organ.

20. A non-transitory program storage device readable by a machine tangibly embodying a program of instructions executable by the machine to perform a method for monitoring, the storage device comprising:
(a) computer code for transmitting a stimulus signal to a neurological organ, said stimulus signal comprising a rhythmic stimulus, said neurological organ comprising a brain;
(b) computer code for receiving a response signal from said neurological organ, said response signal comprising a cortical evoked response;
(c) computer code for transmitting said response signal to a data storage portion, said transmitting said response signal being operable to transmit with a conventional communication protocol, said data storage portion comprising a database, said database being operable to receive and send signals wirelessly;
(d) computer code for receiving said response signal on a display portion, said display portion comprising a portable electronic device, said display portion further comprising a signal parameter, said signal parameter comprising an alert portion; and
(e) computer code for monitoring and manipulating said response signal to optimize at least one cognitive parameter of said neurological organ, said at least one cognitive parameter comprising cognitive performance and behavioral management, said monitoring and manipulating comprising monitoring a real time frequency, said real time frequency comprising about 1 to 10 seconds, said real time frequency further comprising a periodic average, said periodic average comprising about 10 to 60 seconds.
